(19) United States
(12) Patent Application Publication
(10) Pub. No.: US 2007/0225335 A1
(43) Pub. Date: Sep. 27, 2007

(54) Pyrazole Derivatives for Treating Condit Ions Mediated by Activation of the Adeno Sine A2B or A3 Receptor

(76) Inventors: Graham Charles Bloomfield, West Sussex (GB); Catherine Leblanc, West Sussex (GB); Clive McCarthy, Basel (CH); Neil John Press, West Sussex (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/573,273

(22) PCT Filed: Aug. 10, 2005
(86) PCT No.: PCT/EP05/08696

§ 371 Date: Mar. 29, 2007

(30) Foreign Application Priority Data
Aug. 11, 2004 (GB) 0417910.7

(51) Int. Cl. A61K 31/415; A61K 31/4439; C07D 231/10; C07D 401/00

(52) U.S. Cl. 514/341; 514/406; 546/275.4; 548/356.1; 548/365.7

(57) ABSTRACT

Compounds of formula I 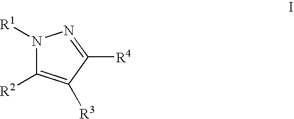
in free or salt form, wherein R¹, R², R³ and R⁴ have the meanings as indicated in the specification, are useful for treating a condition mediated by activation of the adenosine A2b receptor or the adenosine A3 receptor, particularly an inflammatory or obstructive airways disease. Pharmaceutical compositions that contain the compounds and processes for preparing the compounds are also described.

This invention relates to organic compounds, their preparation and use as pharmaceuticals.

In one aspect, the present invention provides compounds of formula I 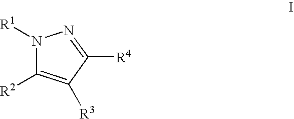
in free or salt form, wherein

R¹ is phenyl optionally substituted by halo, C₁-C₈-alkyl, C₁-C₈-alkoxy, cyano, carboxy, halo-C₁-C₈-alkyl, halo-C₁-C₈-alkoxy, cyano-C₁-C₈-alkyl, carboxy-C₁-C₈-alkyl or aminocarbonyl, or R¹ is a 5- or 6-membered heterocyclic ring containing at least one ring heteroatom selected from the group consisting of nitrogen, oxygen and sulphur, that ring being optionally substituted by C₁-C₈-alkyl, C₁-C₈-alkoxy or one or more oxo groups;

R² is phenyl optionally substituted by halo, C₁-C₈-alkyl, C₁-C₈-alkoxy or morpholinyl, or R² is a 5- or 6-membered heterocyclic ring containing at least one ring heteroatom selected from the group consisting of nitrogen, oxygen and sulphur, that ring being optionally substituted by C₁-C₈-alkyl, C₁-C8-alkoxy or one or more oxo groups;

either R³ and R⁴ are both hydrogen,

or one of R³ and R⁴ is —CO—NR⁵R⁶ and the other is hydrogen;

either R⁵ and R^{b 6} are independently hydrogen; C₁-C₈-alkyl optionally substituted by a 5- or 6-membered heterocyclic ring containing at least one ring heteroatom selected from the group consisting of nitrogen, oxygen and sulphur; C₁-C₈-alkoxy; C₃-C₈-cycloalkyl; a 5- or 6-membered heterocyclic ring containing at least one ring heteroatom selected from the group consisting of nitrogen, oxygen and sulphur; or phenyl optionally substituted by halo, cyano, C₁-C₈-alkyl, C₁-C₈-alkoxy, C₁-C₈-alkylcarbonyl or C₁-C₈-alkoxycarbonyl;
or R⁵ and R⁶ together form 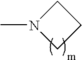
optionally substituted by halo, C₁-C₈-alkyl, C₁-C₈-alkoxy or cyano; and
m is an integer from 0 to 5.

Terms used in the specification have the following meanings:

“Halo” or “halogen” as used herein may be fluorine, chlorine, bromine or iodine. Preferably halo is fluorine or chlorine.

“C₁-C₈-alkyl” as used herein denotes straight chain or branched alkyl having 1 to 8 carbon atoms. Preferably C₁-C₈-alkyl is C₁-C₄-alkyl.

“C₁-C₈-alkoxy” as used herein denotes straight chain or branched alkoxy having 1 to 8 carbon atoms. Preferably C₁-C₈-alkoxy is C₁-C₄-alkoxy.

“C₃-C₈-cycloalkyl” as used herein denotes cycloalkyl having 3 to 8 ring carbon atoms, for example a monocyclic group such as a cyclopropyl, cyclobutyl, cyclopentyl, cyclohexyl, cycloheptyl or cyclooctyl, any of which can be substituted by one or more, usually one or two, C₁-C₄-alkyl groups, or a bicyclic group such as bicycloheptyl or bicyclooctyl. Preferably “C₃-C₈-cycloalkyl” is cyclopropyl, cyclobutyl, cyclopentyl, cyclohexyl, cycloheptyl or cyclooctyl.

“Halo-C₁-C₈-alkyl” and “halo-C₁-C₈-alkoxy” as used herein denote C₁-C₈-alkyl or C₁-C₈-alkoxy respectively as hereinbefore defined substituted at one, two, three or more positions by halo as hereinbefore defined. Preferably halo-C₁-C₈-alkyl and halo-C₁-C₈-alkoxy are halo-C₁-C₄-alkyl and halo-C₁-C₄-alkoxy respectively.

“Cyano-C₁-C₈-alkyl” and “carboxy-C₁-C₈-alkyl” as used herein denote C₁-C₈-alkyl as hereinbefore defined substituted at one, two, three or more positions by cyano or carboxy respectively. Preferably cyano-C₁-C₈-alkyl and carboxy-C₁-C₈-alkyl are cyano-C₁-C₄-alkyl and carboxy-C₁-C₄-alkyl respectively.

“C₁-C₈-alkylcarbonyl” and “C₁-C₈-alkoxycarbonyl” as used herein denote C₁-C₈-alkyl or C₁-C₈-alkoxy respectively as hereinbefore defined attached by a carbon atom to a carbonyl group. Preferably C₁-C₈-alkylcarbonyl and C₁-C₈-alkoxycarbonyl are C₁-C₄-alkylcarbonyl and C₁-C₄-alkoxycarbonyl respectively.

“Aminocarbonyl” as used herein denotes an amino group attached to a carbonyl group.

“5- or 6-membered heterocyclic ring containing at least one ring heteroatom selected from the group consisting of nitrogen, oxygen and sulphur” as used herein may be, for example, pyrrole, pyrazole, imidazole, triazole, tetrazole, thiadiazole, oxazole, isoxazole, isothiazole, oxadiazole, pyridine, pyrazine, pyridazine, pyrimidine, piperazine, morpholino, triazine, oxazine, furan, thiophene or thiazole. Preferred heterocyclic rings include dioxo-tetrahydro-thiophenyl/sulfolanyl, pyridyl and furyl.

“Optionally substituted” means the group referred to can be substituted at one or more positions by any one or any combination of the radicals listed thereafter.

Throughout this specification and in the claims that follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

Preferred compounds of formula I in free or salt form are those where

R¹ is phenyl substituted by halo, C₁-C₈-alkyl or C₁-C₈-alkoxy, or R¹ is a 5-membered heterocyclic ring containing at least one sulphur atom, that ring being optionally substituted by one or more oxo groups;

R² is phenyl optionally substituted by halo or C₁-C₈-alkoxy, or R² is a 6-membered heterocyclic ring containing at least one nitrogen atom;

either R³ and R⁴ are both hydrogen, or

one of R³ and R⁴ is —CO—NR⁵R^{6,} and the other is hydrogen; and

R⁵ and R⁶ are independently hydrogen, C₁-C₈-alkyl optionally substituted by a 5- or 6-membered heterocyclic ring containing at least one nitrogen and/or oxygen atom; C₃-C₈-cycloalkyl; a 5- or 6-membered heterocyclic ring containing at least one nitrogen atom; or phenyl optionally substituted by halo, cyano, C₁-C₈-alkoxy or C₁-C₈-alkylcarbonyl.

Especially preferred compounds of formula I in free or salt form are those where R¹ is phenyl substituted by halo, particularly halo meta to the carbon atom attached to the indicated pyrazole ring, C₁-C₄-alkyl or C₁-C₄-alkoxy, or R¹ is a 5-membered heterocyclic ring containing at least one sulphur atom, that ring being optionally substituted by one or more oxo groups;

R² is phenyl optionally substituted by halo or C₁-C₄-alkoxy, or R² is a 6-membered heterocyclic ring containing at least one nitrogen atom;

either R³ and R⁴ are both hydrogen, or

one of R³ and R⁴ is —CO—NR⁵R⁶, and the other is hydrogen; and

R⁵ and R⁶ are independently hydrogen, C₁-C₄-alkyl optionally substituted by a 5- or 6-membered heterocyclic (preferably unsaturated) ring containing at least one nitrogen and/or oxygen atom; C₃-C₆-cycloalkyl; a 5- or 6-membered heterocyclic (preferably unsaturated) ring containing at least one nitrogen atom; or phenyl optionally substituted by halo, cyano, C₁-C₄-alkoxy or C₁-C₄-alkylcarbonyl.

Especially preferred specific compounds of formula I are those described hereinafter in the Examples.

The compounds represented by formula I are capable of forming acid addition salts, particularly pharmaceutically acceptable acid addition salts. Pharmaceutically acceptable acid addition salts of the compound of formula I include those of inorganic acids, for example, hydrohalic acids such as hydrofluoric acid, hydrochloric acid, hydrobromic acid or hydroiodic acid, nitric acid, sulfuric acid, phosphoric acid; and organic acids, for example aliphatic monocarboxylic acids such as formic acid, acetic acid, trifluoroacetic acid, propionic acid and butyric acid, aliphatic hydroxy acids such as lactic acid, citric acid, tartaric acid or malic acid, dicarboxylic acids such as maleic acid or succinic acid, aromatic carboxylic acids such as benzoic acid, p-chlorobenzoic acid, diphenylacetic acid or triphenylacetic acid, aromatic hydroxy acids such as o-hydroxybenzoic acid, p-hydroxybenzoic acid, 1-hydroxynaphthalene-2-carboxylic acid or 3-hydroxynaphthalene-2-carboxylic acid, and sulfonic acids such as methanesulfonic acid or benzenesulfonic acid. These salts may be prepared from compounds of formula I by known salt-forming procedures.

Compounds of formula I which contain acidic, e.g. carboxyl, groups, are also capable of forming salts with bases, in particular pharmaceutically acceptable bases such as those well known in the art; suitable such salts include metal salts, particularly alkali metal or alkaline earth metal salts such as sodium, potassium, magnesium or calcium salts, or salts with ammonia or pharmaceutically acceptable organic amines or heterocyclic bases such as ethanolamines, benzylamines or pyridine. These salts may be prepared from compounds of formula I by known salt-forming procedures.

In those compounds where there is an asymmetric carbon atom the compounds exist in individual optically active isomeric forms or as mixtures thereof, e.g. as racemic or diastereomeric mixtures. The present invention embraces both individual optically active R and S isomers as well as mixtures, e.g. racemic or diastereomeric mixtures, thereof.

The invention provides, in another aspect, a method of preparing a compound of formula I in free or salt form which comprises
(i) (A) for the preparation of compounds of formula I wherein R³ and R⁴ are both hydrogen, reacting a compound of formula II 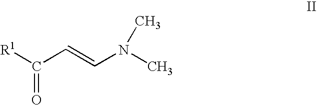
wherein R¹ is as hereinbefore defined, with a compound of formula III 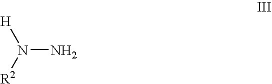
wherein R² is as hereinbefore defined;
(B) for the preparation of compounds of formula I wherein R³ is —CO—NR⁵R⁶ and R⁴ is hydrogen, reacting a compound of formula IV 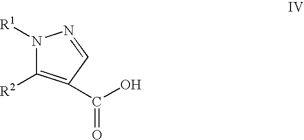
wherein R¹ and R² are as hereinbefore defined, or an amide-forming derivative thereof, with a compound of formula V 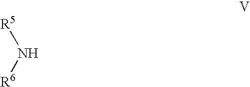
wherein R⁵ and R⁶ are as hereinbefore defined; or
(C) for the preparation of compounds of formula I wherein R³ is hydrogen and R⁴ is —CO—NR⁵R⁶, reacting a compound of formula VI 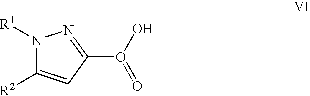
wherein R¹ and R² are as hereinbefore defined, or an amide-forming derivative thereof, with a compound of formula V wherein R⁵ and R⁶ are as hereinbefore defined; and
(ii) recovering the resultant compound of formula I in free or salt form.

Process variant (A) may be carried out using known procedures for reacting enamine compounds with hydrazine derivatives, or analogously e.g. as hereinafter described in the Examples. The reaction is conveniently carried out using an organic solvent, for example dimethylformamide. Suitable reaction temperatures are elevated temperatures, for example from 70° C. to 100° C., but preferably about 90° C.

Process variant (B) may be carried out using known procedures for reacting carboxylic acids (or amide-forming derivatives thereof such as acid halide derivatives) with amines, or analogously e.g. as hereinafter described in the Examples. The reaction is conveniently carried out using an organic solvent, for example dimethylformamide, in the presence of one or more coupling agents, for example O-(7-azabenzotriazol-1-yl)-1,1,3-,3-tetramethyluronium hexafluoro-phosphate (HATU), and a base, for example diisopropylethylamine (DIPEA). Suitable reaction temperatures are from 10° C. to 40° C., for example room temperature.

Process variant (C) may be carried out using known procedures for reacting carboxylic acids (or amide-forming derivatives thereof such as acid halide derivatives) with amines, or analogously e.g. as hereinafter described in the Examples. The reaction is conveniently carried out using an organic solvent, for example dimethylformamide, in the presence of one or more coupling agents, for example O-(7-azabenzotriazol-1-yl)-1,1,3-,3-tetramethyluronium hexafluoro-phosphate (HATU), and a base, for example diisopropylethylamine (DIPEA). Suitable reaction temperatures are from 10° C. to 40° C., for example room temperature.

Compounds of formula II may be prepared by reacting the corresponding acetyl compound with dimethylformamide dimethylacetal. The reaction is conveniently carried out using an organic solvent, for example toluene. Suitable reaction temperatures are elevated temperatures, for example from 70° C. to 100° C., but preferably about 90° C.

Compounds of formula III are either commercially available or may be obtained by known procedures for preparing hydrazines.

Compounds of formula IV may be prepared by hydrolysing the corresponding ester of formula 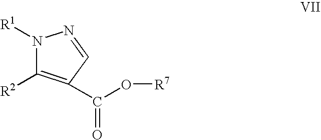
wherein R¹ and R² are as hereinbefore defined and R⁷ is C₁-C₈-alkyl. Hydrolysis is conveniently carried out using known procedures, for example using an alkali metal hydroxide such as sodium hydroxide. Suitable reaction temperatures are from 10° C. to reflux, but preferably reflux.

Compounds of formula V are either commercially available or may be obtained by known procedures for preparing amines.

Compounds of formula VI may be prepared by hydrolysing the corresponding ester of formula VIII 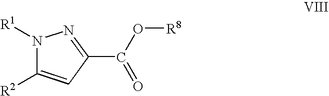
wherein R¹ and R² are as hereinbefore defined and R⁸ is C₁-C₈-alkyl. Hydrolysis is conveniently carried out using known procedures, for example using an alkali metal hydroxide such as sodium hydroxide. Suitable reaction temperatures are from 10° C. to 60° C., but preferably about 60° C.

Compounds of formula VII may be prepared by reacting a compound of formula IX 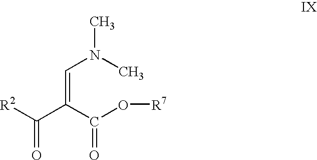
wherein R² and R⁷ are as hereinbefore defined, with a compound of formula X 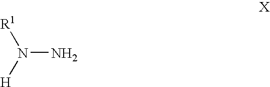
wherein R¹ is as hereinbefore defined. The reaction is conveniently carried out in an acidic solution, for example glacial acetic acid. Suitable reaction temperatures are elevated temperatures, for example from 70° C. to 120° C., but preferably reflux temperature.

Compounds of formula VIII may be prepared by reacting a compound of formula XI 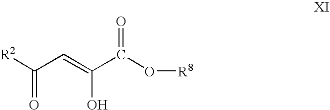
wherein R² and R⁸ are as hereinbefore defined, with a compound of formula X wherein R¹ is as hereinbefore defined. The reaction is conveniently carried out in an acidic solution, for example glacial acetic acid. Suitable reaction temperatures are elevated temperatures, for example from 70° C. to 120° C., but preferably reflux temperature.

Compounds of formula IX may be prepared by reacting a compound of formula XII 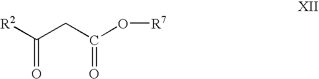
wherein R² and R⁷ are as hereinbefore defined, with dimethylformamide dimethylacetal. The reaction is conveniently carried out using an organic solvent, for example toluene. Suitable reaction temperatures are elevated temperatures, for example from 70° C. to 120° C., but preferably reflux temperature.

Compounds of formula X are either commercially available or may be obtained by known procedures for preparing hydrazines.

Compounds of formula XI may be prepared by reacting a compound of formula XIII 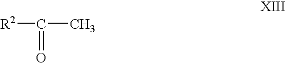
wherein R² is as hereinbefore defined, with an appropriately alkylated oxalate in a basic solution, for example containing sodium methoxide. The reaction is conveniently carried out using an organic solvent, for example methanol. Suitable reaction temperatures are from 10° C. to 50° C., for example room temperature.

Compounds of formula XII, for example ethyl isonicotinoylacetate, are commercially available or may be prepared using methods analogous to those used to prepare ethyl isonicotinoyl-acetate.

Compounds of formula XIII are either commercially available or may be obtained by known procedures for preparing ketones.

Compounds of formula I in free form may be converted into salt form, and vice versa, in a conventional manner. The compounds in free or salt form can be obtained in the form of hydrates or solvates containing a solvent used for crystallisation. Compounds of formula I can be recovered from reaction mixtures and purified in a conventional manner. Isomers, such as enantiomers, may be obtained in a conventional manner, e.g. by fractional crystallisation or asymmetric synthesis from correspondingly asymmetrically substituted, e.g. optically active, starting materials.

Compounds of formula I and their pharmaceutically acceptable salts are useful as pharmaceuticals. In particular, they exhibit inhibition of adenosine A2b receptor activation, i.e. they act as A2b receptor antagonists. Moreover, in general they selectively inhibit activation of A2b receptor over the adenosine A1 and A2a receptors. Their inhibitory properties may be demonstrated in the following test procedures:

Adenosine A2b Receptor Reporter Gene Assay

a) Culturing of Chinese Hamster Ovary (CHO) A2b Cell Line

CHO cells transfected with a Luciferase-expressing reporter plasmid (pCRE-LUCI) and with a plasmid carrying the human adenosine A2b receptor structural gene (pA2bRCV) are routinely cultured in Dulbecco's Modified Eagle Medium (DMEM)—supplemented with 10% v/v fetal calf serum (FCS), 2 mM L-glutamine, 0.4 mg/ml L-proline, 1 nM sodium selenite, 0.5 mg/ml Hygromycin B and 1 mg/ml Geneticin—at 37° C., 5% CO₂ and 100% humidity. The cells are left to grow to confluence for 4-5 days. The cells obtained are passaged using trypsin/EDTA and split at a ratio of 1 in 5.

b) Preparation of cells for assay

Prior to the assay, the CHO-A2b cells are plated onto white 96-well View Plate tissue culture plates (Packard) at a density of 50,000 cells per well in 50 μl of DMEM, and the plates are incubated at 37° C., 5% CO₂ and 100% humidity.

c) Preparation of Reference and Test Compounds

10 mM solutions of the reference compound, Xanthine Amine Cogener (XAC), and the test compound in dimethyl sulfoxide (DMSO) are prepared. The solutions are further diluted with DMSO to 100 μM, then diluted to 10 μM, and finally to 250 nM or 2.5 μM with Assay Buffer (DMEM Phenol Red-free tissue culture media supplemented with 10 μM Rolipram and 10 U/ml adenosine deaminase (ADA). The resulting solutions (40 μl) are added to the cells in the appropriate wells, the final concentration per well being 100 nM or 1 μM, and the plates are incubated at 37° C., 5% CO₂ and 100% humidity.

d) Luciferase Reporter Gene Assay

5′-N-ethylcarboxamidoadenosine (NECA), an adenosine A2b agonist, is prepared as a 10 nM solution in DMSO and then diluted to 100 μM with Assay Buffer. This solution is serially diluted in Assay Buffer to give a series of 10 NECA concentrations from 100 to 0.01 μM. 10 μl portions of the resulting NECA solutions are added to the mixtures of CHO-A2b cells and reference or test compound solutions prepared as described above (preincubated for 30 minutes), final concentrations ranging from 10 to 0.0005 μM per well. The cells are incubated at 37° C., 5% CO₂ and 100% humidity for 3 hours to induce release of cAMP, which then binds to cAMP binding protein (CBP) and the resulting complex interacts with the reporter plasmid to express Luciferase. 100 μl of Steady-Glo, a Luciferase assay substrate from Promega, is added to all wells to lyse the cells and generate luminescence in proportion to the amount of Lucifrease produced. The plates are left for a minimum of 5 minutes before being read on the luminescence program of a Topcount NXT microplate scintillation counter (ex Packard). Concentration—response curves are plotted from the luminescence data using Activitybase software and K_{B} values for the antagonists under test are calculated from the shifts of the curve at a particular concentration (K_{B}=[antagonist]/(concentration ratio−1)

Compounds of the Examples hereinbelow have K_{B} values below 1.5 μM in the reporter gene assay. For example, the compounds of Examples 4, 14, 24, 33 and 38 have K_{B} values of 0.139, 0.224, 0.041, 0.188 and 0.240 μM respectively.

In general, compounds of formula I in free or pharmaceutically acceptable salt form also exhibit inhibition of adenosine A3 receptor activation, which may be demonstrated in the adenosine A3 receptor assay described in WO 99/64418.

Having regard to their inhibition of adenosine A2b receptor activation, and, in general, their inhibition of adenosine A3 receptor activation, compounds of formula I in free or pharmaceutically acceptable salt form, hereinafter alternately referred to as “agents of the invention”, are useful in the treatment of conditions which are mediated by the activation of the adenosine A2b receptor or the adenosine A3 receptor, particularly inflammatory or allergic conditions. Treatment in accordance with the invention may be symptomatic or prophylactic. Accordingly, agents of the invention are useful in the treatment of inflammatory or obstructive airways diseases, resulting, for example, in reduction of tissue damage, airways inflammation, bronchial hyperreactivity, remodelling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, i.e. therapy for or intended to restrict or abort symptomatic attack when it occurs, for example anti-inflammatory (e.g. corticosteroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e. at a time normally substantially distant form any previously administered symptomatic asthma therapy.

Other inflammatory or obstructive airways diseases and conditions to which the present invention is applicable include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Having regard to their anti-inflammatory activity, in particular in relation to inhibition of eosinophil activation, agents of the invention are also useful in the treatment of eosinophil related disorders, e.g. eosinophilia, in particular eosinophil related disorders of the airways (e.g. involving morbid eosinophilic infiltration of pulmonary tissues) including hypereosinophilia as it effects the airways and/or lungs as well as, for example, eosinophil-related disorders of the airways consequential or concomitant to Löffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

Agents of the invention are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, and other inflammatory or allergic conditions of the skin.

Agents of the invention may also be used for the treatment of other diseases or conditions, in particular diseases or conditions having an inflammatory component, for example, treatment of diseases and conditions of the eye such as conjunctivitis, keratoconjunctivitis sicca, and vernal conjunctivitis, diseases affecting the nose including allergic rhinitis, and inflammatory disease in which autoimmune reactions are implicated or having an autoimmune component or aetiology, including autoimmune haematological disorders (e.g. haemolytic anaemia, aplastic anaemia, pure red cell anaemia and idiopathic thrombocytopenia), systemic lupus erythematosus, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary billiary cirrhosis, uveitis (anterior and posterior), keratoconjunctivitis sicca and vernal keratoconjunctivitis, interstitial lung fibrosis, psoriatic arthritis and glomerulonephritis (with and without nepbrotic syndrome, e.g. including idiopathic nephrotic syndrome or ninal change nephropathy).

Other diseases or conditions which may be treated with agents of the invention include diabetes, e.g. diabetes mellitus type I (juvenile diabetes) and diabetes mellitus type II, diarrheal diseases, ischemia/reperfusion injuries, retinopathy, such as diabetic retinopathy or hyperbaric oxygen-induced retinopathy, and conditions characterised by elevated intraocular pressure or secretion of ocular aqueous humor, such as glaucoma.

The effectiveness of an agent of the invention in inhibiting inflammatory conditions, for example in inflammatory airways diseases, may be demonstrated in an animal model, e.g. a mouse or rat model, of airways inflammation or other inflammatory conditions, for example as described by Szarka et al, J. Immunol. Methods (1997) 202:49-57; Renzi et al, Am. Rev. Respir. Dis. (1993) 148:932-939; Tsuyuki et al., J. Clin. Invest. (1995) 96:2924-2931; and Cernadas et al (1999) Am. J. Respir. Cell Mol. Biol. 20:1-8.

The agents of the invention are also useful as co-therapeutic agents for use in combination with other drug substances such as anti-inflammatory, bronchodilatory, antihistamine or anti-tussive drug substances, particularly in the treatment of obstructive or inflammatory airways diseases such as those mentioned hereinbefore, for example as potentiators of therapeutic activity of such drugs or as a means of reducing required dosaging or potential side effects of such drugs. An agent of the invention may be mixed with the other drug substance in a fixed pharmaceutical composition or it may be administered separately, before, simultaneously with or after the other drug substance.

Such anti-inflammatory drugs include steroids, in particular glucocorticosteroids such as budesonide, beclamethasone dipropionate, fluticasone propionate, ciclesonide or mometasone furoate, or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/35668, WO 03/48181, WO 03/62259, WO 03/64445, WO 03/72592, WO 04/39827 and WO 04/66920; non-steroidal glucocorticoid receptor agonists, such as those described in DE 10261874, WO 00/00531, WO 02/10143, WO 03/82280, WO 03/82787, WO 03/86294, WO 03/104195, WO 03/101932, WO 04/05229, WO 04/18429, WO 04/19935 and WO 04/26248; LTB4 antagonists such as BIIL 284, CP-195543, DPC11870, LTB4 ethanolamide, LY 293111, LY 255283, CGS025019C, CP-195543, ONO-4057, SB 209247, SC-53228 and those described in U.S. Pat. No. 5,451,700; LTD4 antagonists such include montelukast, pranlukast, zafirlukast, accolate, SR2640, Wy-48,252, ICI 198615, MK-571, LY-171883, Ro 24-5913 and L-648051; dopamine receptor agonists such as cabergoline, bromocriptine, ropinirole and 4-hydroxy-7-[2-[[2-[[3-(2-phenylethoxy)propyl]sulfonyl]ethyl]-amino]ethyl]-2(3H)-benzothiazolone and pharmaceutically acceptable salts thereof (the hydrochloride being Viozan®-AstraZeneca); PDE4 inhibitors such cilomilast (Ariflo® GlaxoSmithKline), Roflumilast (Byk Gulden),V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SelCID(™) CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805.

Such bronchodilatory drugs include anticholinergic or antimuscarinic agents, in particular ipratropium bromide, oxitropium bromide, tiotropium salts and CHF 4226 (Chiesi), and glycopyrrolate, but also those described in EP 424021, U.S. Pat. No. 3,714,357, U.S. Pat. No. 5,171,744, WO 01/04118, WO 02/00652, WO 02/51841, WO 02/53564, WO 03/00840, WO 03/33495, WO 03/53966, WO 03/87094, WO 04/018422 and WO 04/05285; and beta (β)-2-adrenoceptor agonists such as beta-2 adrenoceptor agonists such as albuterol (salbutamol), metaproterenol, terbutaline, salmeterol fenoterol, procaterol, and especially, formoterol, carmoterol and pharmaceutically acceptable salts thereof, and compounds (in free or salt or solvate form) of formula I of WO 0075114, which document is incorporated herein by reference, preferably compounds of the Examples thereof, especially a compound of formula 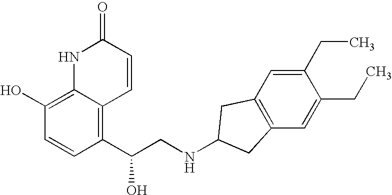
and pharmaceutically acceptable salts thereof, as well as compounds (in free or salt or solvate form) of formula I of WO 04/16601, and also compounds of EP 1440966, JP 05025045, WO 93/18007, WO 99/64035, US 2002/0055651, WO 01/42193, WO 01/83462, WO 02/66422, WO 02/70490, WO 02/76933, WO 03/24439, WO 03/42160, WO 03/42164, WO 03/72539, WO 03/91204, WO 03/99764, WO 04/16578, WO 04/22547, WO 04/32921, WO 04/33412, WO 04/37768, WO 04/37773, WO 04/37807, WO 04/39762, WO 04/39766, WO 04/45618 WO 04/46083, WO 04/80964, EP1460064, WO 04/087142, WO 04/089892, EP 01477167, US 2004/0242622, US 2004/0229904, WO 04/108675, WO 04/108676, WO 05/033121, WO 05/040103 and WO 05/044787.

Co-therapeutic antihistamine drug substances include cetirizine hydrochloride, acetamino-phen, clemastine fumarate, promethazine, loratidine, desloratidine, diphenhydramine and fexofenadine hydrochloride, activastine, astemizole, azelastine, ebastine, epinastine, mizolastine and tefenadine as well as those disclosed in WO 03/099807, WO 04/026841, JP 2004107299.

Combinations of agents of the invention and one or more steroids, beta-2 agonists, PDE4 inhibitors or LTD4 antagonists may be used, for example, in the treatment of COPD or, particularly, asthma. Combinations of agents of the invention and anticholinergic or antimuscarinic agents, PDE4 inhibitors, dopamine receptor agonists or LTB4 antagonists may be used, for example, in the treatment of asthma or, particularly, COPD.

Other useful combinations of agents of the invention with anti-inflammatory drugs are those with other antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770), CCR-5 antagonists described in U.S. Pat. No. 6,166,037 (particularly claims 18 and 19), WO 00/66558 (particularly claim 8), and WO 00/66559 (particularly claim 9), WO 04/018425 and WO 04/026873.

In accordance with the foregoing, the invention also provides a method for the treatment of a condition mediated by activation of the adenosine A2b receptor, and/or the adenosine A3 receptor, for example an inflammatory or allergic condition, particularly an inflammatory or obstructive airways disease, which comprises administering to a subject, particularly a human subject, in need thereof a compound of formula I in free form or in the form of a pharmaceutically acceptable salt. In another aspect the invention provides a compound of formula I, in free form or in the form of a pharmaceutically acceptable salt, for use in the manufacture of a medicament for the treatment of a condition mediated by activation of the adenosine A2b receptor, and/or the adenosine A3 receptor, particularly an inflammatory or obstructive airways disease.

The agents of the invention may be administered by any appropriate route, e.g. orally, for example in the form of a tablet or capsule; parenterally, for example intravenously; by inhalation, for example in the treatment of inflammatory or obstructive airways disease; intranasally, for example in the treatment of allergic rhinitis; topically to the skin, for example in the treatment of atopic dermatitis; or rectally, for example in the treatment of inflammatory bowel disease.

In a further aspect, the invention also provides a pharmaceutical composition comprising a compound of formula I in free form or in the form of a pharmaceutically acceptable salt, optionally together with a pharmaceutically acceptable diluent or carrier therefor. The composition may contain a co-therapeutic agent such as an anti-inflammatory, bronchodilatory or antihistamine drug as hereinbefore described. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g. patches. Compositions for inhalation may comprise aerosol or other atomizable formulations or dry powder formulations.

When the composition comprises an aerosol formulation, it preferably contains, for example, a hydro-fluoro-alkane (HFA) propellant such as HFA134a or HFA227 or a mixture of these, and may contain one or more co-solvents known in the art such as ethanol (up to 20% by weight), and/or one or more surfactants such as oleic acid or sorbitan trioleate, and/or one or more bulking agents such as lactose. When the composition comprises a dry powder formulation, it preferably contains, for example, the compound of formula I having a particle diameter up to 10 microns, optionally together with a diluent or carrier, such as lactose, of the desired particle size distribution and a compound that helps to protect against product performance deterioration due to moisture, such as magnesium stearate ( e.g. 0.05 to 1.5%). When the composition comprises a nebulised formulation, it preferably contains, for example, the compound of formula I either dissolved, or suspended, in a vehicle containing water, a co-solvent such as ethanol or propylene glycol and a stabiliser, which may be a surfactant.

Dosages of compounds of formula I employed in practising the present invention will of course vary depending, for example, on the particular condition to be treated, the effect desired and the mode of administration. In general, suitable daily dosages for administration by inhalation are of the order of 0.005 to 10 mg, while for oral administration suitable daily doses are of the order of 0.05 to 100 mg.

The invention is illustrated by the following Examples.

### EXAMPLES 1-40

Compounds of formula I

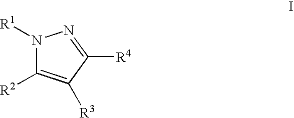

are shown in the following table. Methods for preparing such compounds are described hereinafter. The table also shows mass spectrometry, MH+ {ESMS), data. The Examples are in free form.

| Ex. | R¹ | R² | R³ | R⁴ | MH⁺ |
|---|---|---|---|---|---|
| 1 | 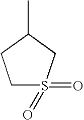 | 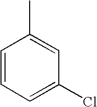 | H | H | 279.2 |
| 2 | 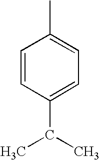 | 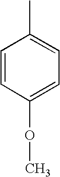 | H | H | 293.2 |
| 3 | 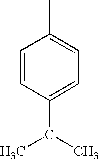 | 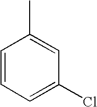 | H | H | 297.2 |
| 4 | 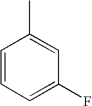 | 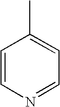 | 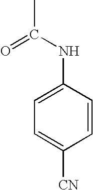 | H | 384.2 |
| 5 | 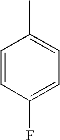 | 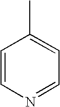 | 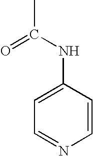 | H | 360.2 |
| 6 | 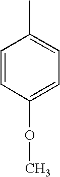 | 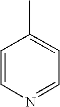 | 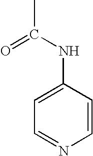 | H | 371.8 |
| 7 | 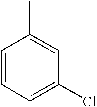 | 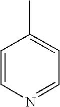 | 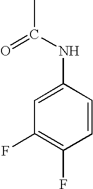 | H | 411.2 |
| 8 | 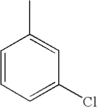 | 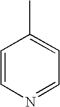 | 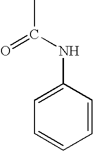 | H | 375.2 |
| 9 | 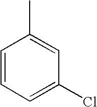 | 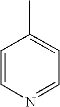 | 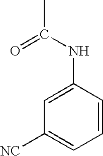 | H | 354.8 |
| 10 | 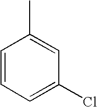 | 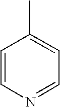 | 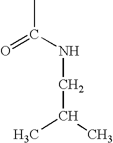 | H | 354.8 |
| 11 | 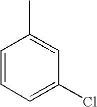 | 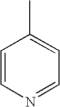 | 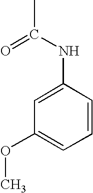 | H | 404.8 |
| 12 | 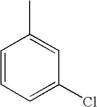 | 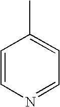 | 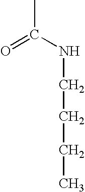 | H | 354.8 |
| 13 | 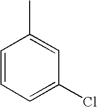 | 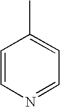 | 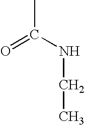 | H | 326.8 |
| 14 | 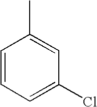 | 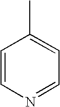 | 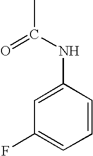 | H | 392.7 |
| 15 | 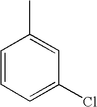 | 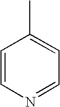 | 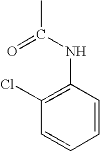 | H | 410.7 |
| 16 | 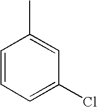 | 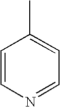 | 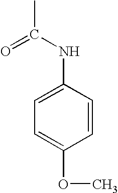 | H | 405.2 |
| 17 | 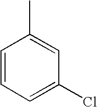 | 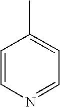 | 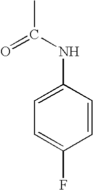 | H | 393.2 |
| 18 | 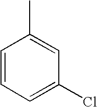 | 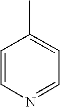 | 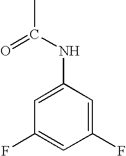 | H | 411.1 |
| 19 | 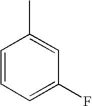 | 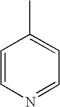 | 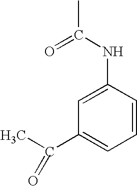 | H | 401.2 |
| 20 | 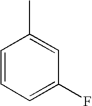 | 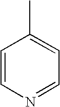 | 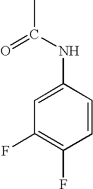 | H | 395.1 |
| 21 | 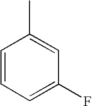 | 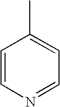 | 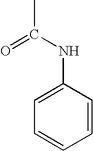 | H | 359.2 |
| 22 | 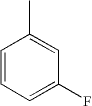 | 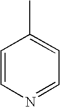 | 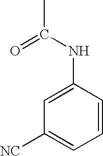 | H | 384.2 |
| 23 | 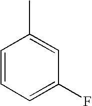 | 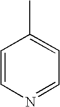 | 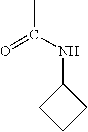 | H | 337.2 |
| 24 | 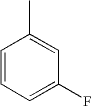 | 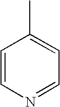 | 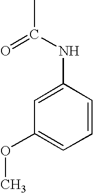 | H | 389.2 |
| 25 | 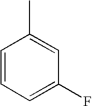 | 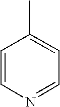 | 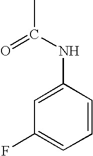 | H | 377.2 |
| 26 | 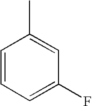 | 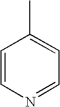 | 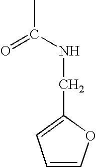 | H | 363.2 |
| 27 | 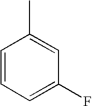 | 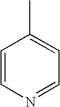 | 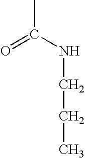 | H | 324.9 |
| 28 | 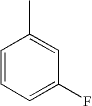 | 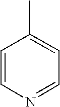 | 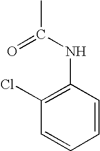 | H | 392.7 |
| 29 | 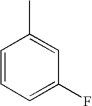 | 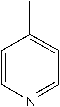 | 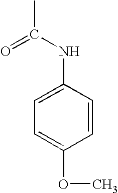 | H | 389.2 |
| 30 | 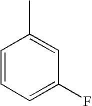 | 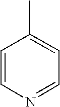 | 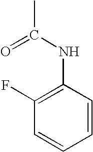 | H | 377.2 |
| 31 | 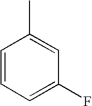 | 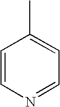 | 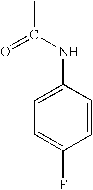 | H | 377.2 |
| 32 | 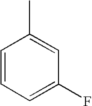 | 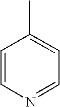 | 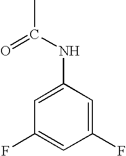 | H | 395.2 |
| 33 | 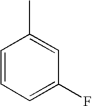 | 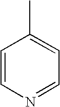 | H | 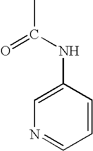 | 360.1 |
| 34 | 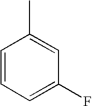 | 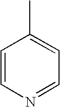 | H | 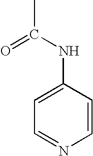 | 360.1 |
| 35 | 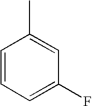 | 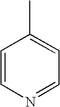 | H | 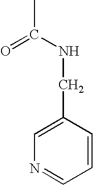 | 374.1 |
| 36 | 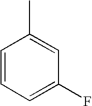 | 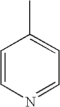 | H | 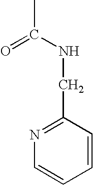 | 374.1 |
| 37 | 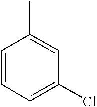 | 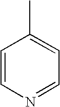 | H | 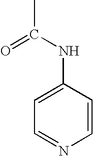 | 376.1 |
| 38 | 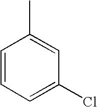 | 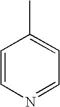 | H | 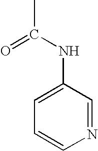 | 376.1 |
| 39 | 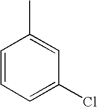 | 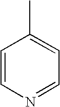 | H | 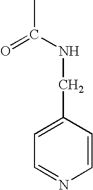 | 390.1 |
| 40 | 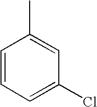 | 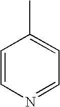 | H | 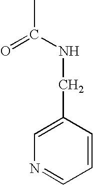 | 390.1 |

Preparation of Specific Examples:

### Example 1

### 5-(3-Chloro-phenyl)-1-(1,1-dioxo-tetrahydro-thiophen-3yl)-1H-pyrazole

A solution of 3-chloroacetophenone (1.54 g, 10 mmol) in toluene (10 ml) is treated with dimethylformamide dimethylacetal (5.4 ml, 40 mmol). The mixture is stirred at 100° C. overnight, followed by removal of the solvent in vacuo to give (E)-1-(3-chloro-phenyl)-3-dimethylamino-propenone. MH+ (ESMS): 209.6

A solution of the enamine intermediate (0.02 g, 0.1 mmol) in ethanol (0.5 ml) is treated with (1,1-dioxo-tetrahydro-thiophen-3yl)-hydrazine (0.015 g, 0.1 mmol) in dimethylformamide (DMF) (0.5 ml). The mixture is stirred at 90° C. overnight. The solvent is removed in vacuo and the residue is purified by prep LCMS (Liquid chromatography-mass spectroscopy) to give the title compound. MH+ (ESMS): 279.2

The compounds of the Examples 2 and 3 are prepared using procedures analogous to that used in Example 1.

### Example 4

### 1-(3-Fluoro-phenyl)-5-pyridin-4-yl-1H-pyrazole4-carboxylic acid (4-cyano-phenyl)-amide

4a) 3-Dimethylamino-2-(pyridine-4-carbonyl)-acrylic acid ethyl ester

A solution of dimethylformamide dimethylacetal (5.57 ml, 41.45 mmol) in toluene (25 ml) is added in one portion to a solution of ethyl isonicotinoylacetate (5 g, 25.9 mmol) in toluene (25 ml). The mixture is refluxed for one hour followed by removal of the solvent in vacuo to give the crude enamine.

4b) 1-(3-Fluorophenyl)-5-pvridin-4-yl-1H-pyrazole-4-carboxylic acid ethyl ester

To 3-Dimethylamino-2-(pyridine-4-carbonyl)-acrylic acid ethyl ester (3.34 g, 13.5 mmol) in glacial acetic acid (30 ml) is added 1-(3-fluorophenyl) hydrazine (13.5 mmol) and the mixture is refluxed overnight. The reaction mixture is poured into water (50 ml) and extracted with chloroform (3×15 ml). The combined organic phases are washed with 5% sodium hydrogen carbonate (2×20 ml), water (2×20 ml) and then dried with magnesium sulphate. The solvent is evaporated and the residue is purified by chromatography using hexane/ethyl acetate (1:1) as eluent to give the titled compound.

4c) 1-(3-Fluorophenyl)-5-pyridin-4-yl-1H-pyrazole-4-carboxylic acid

To 1-(3-Fluorophenyl)-5-pyridin-4-yl-1H-pyrazole-4-carboxylic acid ethyl ester (3.14 g, 10.12 mmol) in 30% aqueous dioxane (60 ml) is added 2.5 N NaOH (12.6 ml). The mixture is stirred at reflux for 1 hour then at 50° C. overnight. The mixture is acidified with 1N HCl (˜36 ml) and the resulting solid is filtered, washed with water and dried in vacuo to give the title compound as a white solid.

4d) 1-(3-Fluoro-phenyl)-5-pyridin-4-yl-1H-pyrazole-4-carboxylic acid (4-cyano-phenyl)-amide

To a suspension of 1-(3-fluorophenyl)-5-pyridin-4-yl-1H-pyrazole-4-carboxylic acid (0.1 g, 0.35 mmol) in DMF (2 ml) is added a solution of diisopropylethylamine (DIPEA) (0.182 g, 1.4 mmol) in DMF (0.5 ml) followed by a solution of HATU (0.268 g, 0.7 mmol) in DMF (0.5 ml). After 40 min at room temperature, a solution of 4-aminobenzonitrile (0.125 g, 1.05 mmol) in DMF (0.5 ml) is added. The mixture is stirred overnight at room temperature. The solvent is removed in vacuo and the residue is purified by prep LCMS to give the title compound. MH+ (ESMS): 384.2

The compounds of the Examples 5 to 32 are prepared using procedures analogous to that used in Example 4.

### Example 33

### 1-(3-Fluoro-phenyl)-5-pyridin-4-yl-1H-pyrazole-3-carboxylic acid pyridin-3-ylamide

33a) (Z)-2-Hydroxyb-4-oxo-4-pyridin-4-yl-but-2-enoic acid methyl ester

A stirred solution of 4-Acetylpyridine (5 g, 41.3 mmol) in dry methanol (100 ml) at room temperature is treated with dimethyloxalate (7.8 g, 66.1 mmol). A solution of sodium methoxide (25% w/v in methanol, 18 ml, 82.6 mmol) is added and stirring continued overnight. The precipitated solid is filtered off, washed with methanol (200 ml) and dried under vacuum to give the titled compound.

33b) 1-(3-Fluorophenyl)-5-pyridin-4-yl-1H-pyrazole-3-carboxylic acid methyl ester

A stirred suspension of (Z)-2-Hydroxy-4-oxo-4-pyridin-4-yl-but-2-enoic acid methylester (1.0 g, 4.8 mmol) in glacial acetic acid (10 ml) is treated with 1-(3-fluorophenyl) hydrazine (0.78 g, 4.8 mmol) and the mixture is refluxed for 7 hours. The solvent is removed in vacuo and the residue is purified by chromatography using hexane/ethyl acetate (1:1) as eluent to give the titled compound.

33c) 1-(3-Fluorophenyl)-5-pyridin-4-yl-1H-pyrazole-3-carboxlic acid

A solution of 1-(3-Fluorophenyl)-5-pyridin-4-yl-1H-pyrazole-3-carboxylic acid methyl ester (0.860 g, 2.89 mmol) in dioxane/water (20 ml, 1/1) is treated with 2.5 N NaOH (2 ml). The mixture is stirred overnight then is acidified with 1N HCl. The resulting solid is filtered and dried in vacuo to give the titled compound.

33d) 1-(3-Fluoro-phenyl)-5-pyridin-4-yl-1H-pyrazole-3-carboxylic acid pyridin-3-ylamide

To a suspension of 1-(3-fluorophenyl)-5-pyridin-4-yl-1H-pyrazole-3-carboxylic acid (0.05 g, 0.18 mmol) and DIPEA (0.09 ml, 0.54 mmol) in DMF (0.5 ml) is added a solution of HATU (0.14 g, 0.36 mmol) in DMF (0.5 ml). After 20 minutes at room temperature, a solution of 3-aminopyridine (0.017 g, 0.18 mmol) in DMF (0.5 ml) is added. The mixture is stirred at room temperature for 4 hours. The solvent is removed in vacuo and the residue is purified by prep LCMS to give the title compound. MH+ (ESMS): 360.1

The compounds of Examples 34 to 40 are prepared using procedures analogous to that used in Example 33.

1. A compound of formula I 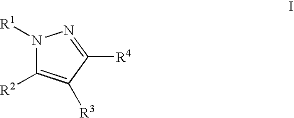
in free or salt form, wherein
R¹ is phenyl optionally substituted by halo, C₁-C₈-alkyl, C₁-C₈-alkoxy, cyano, carboxy, halo-C₁-C₈-alkyl, halo-C₁-C₈-alkoxy, cyano-C₁-C₈-alkyl, carboxy-C₁-C₈-alkyl or aminocarbonyl, or R¹ is a 5- or 6-membered heterocyclic ring containing at least one ring heteroatom selected from the group consisting of nitrogen, oxygen and sulphur, that ring being optionally substituted by C₁-C₈-alkyl, C₁-C₈-alkoxy or one or more oxo groups;
R² is phenyl optionally substituted by halo, C₁-C₈-alkyl, C₁-C₈-alkoxy or morpholinyl, or R² is a 5-or 6-membered heterocyclic ring containing at least one ring heteroatom selected from the group consisting of nitrogen, oxygen and sulphur, that ring being optionally substituted by C₁-C₈-alkyl, C₁-C₈-alkoxy or one or more oxo groups;
either R³ and R⁴ are both hydrogen,
or one of R³ and R⁴ is —CO—NR⁵R⁶ and the other is hydrogen;
either R⁵ and R⁶ are independently hydrogen; C₁-C₈-alkyl optionally substituted by a 5- or 6-membered heterocyclic ring containing at least one ring heteroatom selected from the group consisting of nitrogen, oxygen and sulphur; C₁-C₈-alkoxy; C₃-C₈-cycloalkyl; a 5- or 6- membered heterocyclic ring containing at least one ring heteroatom selected from the group consisting of nitrogen, oxygen and sulphur; or phenyl optionally substituted by halo, cyano, C₁-C₈-alkyl, C₁-C₈-alkoxy, C₁-C₈-alkylcarbonyl or C₁-C₈-alkoxycarbonyl;
or R⁵ and R⁶ together form 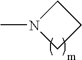
optionally substituted by halo, C₁-C₈-alkyl, C₁-C₈-alkoxy or cyano; and
m is an integer from 0 to 5.

2. A compound according to claim 1, in which
R¹ is phenyl substituted by halo, C₁-C₈-alkyl or C₁-C₈-alkoxy, or R¹ is a 5-membered heterocyclic ring containing at least one sulphur atom, that ring being optionally substituted by one or more oxo groups;
R² is phenyl optionally substituted by halo or C₁-C₈-alkoxy, or R² is a 6-membered heterocyclic ring containing at least one nitrogen atom;
either R³ and R⁴ are both hydrogen, or
one of R³ and R⁴ is —CO—NR⁵R⁶, and the other is hydrogen; and
R⁵ and R⁶ are independently hydrogen, C₁-C₈-alkyl optionally substituted by a 5- or 6-membered heterocyclic ring containing at least one nitrogen and/or oxygen atom; C₃-C₈-cycloalkyl; a 6-membered heterocyclic ring containing at least one nitrogen atom; or phenyl optionally substituted by halo, cyano, C₁-C₈-alkoxy or C₁-C₈-alkylcarbonyl.

3. A compound according to claim 2, in which
R¹ is phenyl substituted by halo, particularly halo meta to the carbon atom attached to the indicated pyrazole ring, C₁-C₄-alkyl or C₁-C₄-alkoxy, or R¹ is a 5-membered heterocyclic ring containing at least one sulphur atom, that ring being optionally substituted by one or more oxo groups;
R² is phenyl optionally substituted by halo or C₁-C₄-alkoxy, or R² is a 6-membered heterocyclic ring containing at least one nitrogen atom;
either R³ and R⁴ are both hydrogen, or
one of R³ and R⁴ is —CO—NR⁵R⁶, and the other is hydrogen; and
R⁵ and R⁶ are independently hydrogen, C₁-C₄-alkyl optionally substituted by a 5- or 6-membered heterocyclic (preferably unsaturated) ring containing at least one nitrogen and/or oxygen atom; C₃-C₆-cycloalkyl; a 5- or 6-membered heterocyclic (preferably unsaturated) ring containing at least one nitrogen atom; or phenyl optionally substituted by halo, cyano, C₁-C₄-alkoxy or C₁-C₄-alkylcarbonyl.

4. A compound of formula I substantially as herein described in any one of the Examples.

5. A compound according to claim 1 for use as a pharmaceutical.

6. A compound according to claim 1 in combination with an anti-inflammatory, bronchodilatory, antihistamine or anti-tussive drug substance, said compound and said drug substance being in the same or different pharmaceutical composition.

7. A pharmaceutical composition comprising as active ingredient a compound according to claim 1, optionally together with a pharmaceutically acceptable diluent or carrier.

8-10. (canceled)

11. A method of preparing a compound of formula I as defined in claim 1 in free or salt form which comprises
(i) (A) for the preparation of compounds of formula I wherein R³ and R⁴ are both hydrogen, reacting a compound of formula II 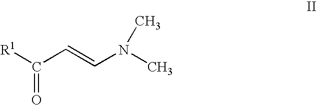
wherein R¹ is as hereinbefore defined, with a compound of formula III 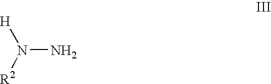
wherein R² is as hereinbefore defined;
(B) for the preparation of compounds of formula I wherein R³ is —CO—NR⁵R⁶ and R⁴ is hydrogen, reacting a compound of formula IV 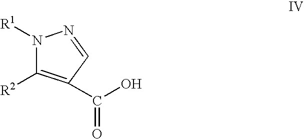
wherein R¹ and R² are as hereinbefore defined, or an amide-forming derivative thereof, with a compound of formula V 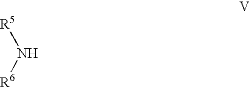
wherein R⁵ and R⁶ are as hereinbefore defined; or
(C) for the preparation of compounds of formula I wherein R³ is hydrogen and R⁴ is CO—NR⁵R⁶, reacting a compound of formula VI 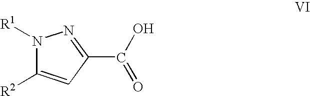
wherein R¹ and R² are as hereinbefore defined, or an amide-forming derivative thereof, with a compound of formula V wherein R⁵ and R⁶ are as hereinbefore defined; and
(ii) recovering the resultant compound of formula I in free or salt form.

12. A method of treating a condition mediated by an adenosine A2b receptor which comprises administering to a subject in need of such a treatment an effective amount of a compound according to claim 1.

13. A method of treating a condition mediated by an adenosine A3 receptor which comprises administering to a subject in need of such a treatment an effective amount of a compound according to claim 1.

14. A method of treating an inflammatory or allergic condition which comprises administering to a subject in need of such a treatment an effective amount of a compound according to claim 1.

15. A method according to claim 14 wherein said inflammatory or allergic condition is an inflammatory or obstructive airways disease.
